(19) United States
(12) Patent Application Publication
(10) Pub. No.: US 2024/0041334 A1
(43) Pub. Date: Feb. 8, 2024

(54) SYSTEMS AND METHODS FOR MEASURING PHYSIOLOGIC VITAL SIGNS AND BIOMARKERS USING OPTICAL DATA

(71) Applicant: BioTrillion, Inc., San Francisco, CA (US)

(72) Inventors: Savan R. Devani, San Francisco, CA (US); Derrick Mo, San Francisco, CA (US); Rishab Gulati, San Francisco, CA (US)

(21) Appl. No.: 18/365,160

(22) Filed: Aug. 3, 2023

Related U.S. Application Data
(60) Provisional application No. 63/395,256, filed on Aug. 4, 2022.

(51) Int. Cl. A61B 5/0205; A61B 5/00; A61B 5/1455; G16H 10/60; G16H 50/70

(52) U.S. Cl. CPC A61B 5/0205; A61B 5/7221; A61B 5/7207; A61B 5/7267; A61B 5/14551; G16H 10/60; G16H 50/70; A61B 5/02125

(57) ABSTRACT

The present disclosure concerns a method and system for remote photoplethysmography (rPPG). The system uses a camera to capture images of a subject and processes these images to monitor physiological parameters such as heart rate and respiratory rate. This is achieved by analyzing subtle changes in skin color that occur due to blood volume changes in the skin. The system allows for contactless, remote, video-based vital signs measurements.

### CROSS-REFERENCE TO RELATED APPLICATIONS

This application claims the benefit of and priority to U.S. Provisional Application No. 63/395,256, filed Aug. 4, 2022, which is hereby incorporated by reference herein in its entirety.

### FIELD OF THE DISCLOSURE

The present disclosure generally relates to non-contact measurement of physiological vital signs and/or digital biomarkers from optical data. More particularly, the present disclosure encompasses remote photoplethysmography (rPPG), transdermal optical imaging (TOI), and the like, to analyze areas of skin of a user in visual media (e.g., video). The visual media can be captured using a wide array of optical recording devices, such as, for example, smartphones, webcams, cameras, camcorders, laptops, tablets, wearable devices with cameras, augmented reality/virtual reality (AR/VR) headsets, or any other device capable of recording images and/or video of a user (or “subject”).

### BACKGROUND

Health monitoring traditionally relies on direct contact methods using dedicated medical and/or wearable devices. These methods are inherently invasive as they require skin contact, and can raise concerns related to hygiene, cost, real-time visibility of health statistics, retrospective analysis, and issues of scalability and mobility.

Technologies such as remote photoplethysmography (rPPG) and transdermal optical imaging (TOI) can facilitate the extraction of vital health metrics from facial videos recorded by common optical devices. However, these techniques present significant limitations. Factors like low spatial and temporal resolution of video data, lighting and motion variations, differences in signal-level strengths, subject-to-camera distance, and variations in skin can compromise the precision of measurements.

Prior technologies fail to accurately extract photoplethysmographic signals due to variations in skin and ambient light. Current rPPG and TOI techniques do not possess a robust, versatile peak detection module capable of accurately determining heart rate, reducing errors in heart rate estimation, and minimizing false negative peaks. Moreover, the scope of computed vital signs from blood flow metrics is limited in existing methods. Further, contemporary systems lack a comprehensive, contactless method for measuring vital signs using video data and specialized modules. The present disclosure addresses these and other challenges.

### SUMMARY

The present disclosure is directed to systems, methods, and computational processes for measuring a subject's vital signs using image and/or video data. This system leverages software modules and mathematical models to extract key health metrics, including but not limited to, heart rate (HR), heart rate variability (HRV), oxygen saturation (SpO2), respiration rate (RR), and blood pressure (BP). Using color channel information in video data, and processed via proprietary methods, the system mitigates rPPG challenges such as lighting variations, motion artifacts, signal-level strengths, subject-to-camera distance, and skin differences, thus resulting in an accurate and reliable output of information.

Furthermore, the systems of the present disclosure enhance the accuracy and precision of measured vital signs, even in non-controlled videos with artifacts introduced by subject motion, environmental lighting, and other dynamic factors. The systems and methods of the present disclosure represent an innovative combination of multiple domains, including optical hardware, computer vision, machine learning, image processing, and physiology, to provide a convenient and user-friendly platform for contactless, remote, video-based vital signs measurement and monitoring.

The systems and methods of the present disclosure are applied across various fields beyond healthcare, including telemedicine, fitness monitoring, stress measurement, and other fields. It will be appreciated that the disclosed features may be combined into many other different systems and/or applications. Various alternatives, modifications, variations, or improvements therein may be subsequently made by those skilled in the art which are also encompassed by the following disclosure and claims herein.

### BRIEF DESCRIPTION OF THE DRAWINGS

The accompanying drawings exemplify the implementations of the present disclosure and, together with the description, serve to explain and illustrate principles of the present disclosure. The drawings are intended to illustrate major features of the exemplary implementations in a diagrammatic manner.

FIG. 1 is a perspective view of a user holding a smartphone recording a video to generate image data to be used according to some implementations of the present disclosure;

FIG. 2 is flow chart for a method of processing at least a portion of the generated image data of FIG. 1 according to some implementations of the present disclosure;

FIG. 3 is an illustration of a preprocessing step of the method of FIG. 2 for a first frame of the image data according to some implementations of the present disclosure;

FIG. 4 is an illustration of a ROI selection step of the method of FIG. 2 for the first frame of the image data according to some implementations of the present disclosure;

FIG. 5 is an illustration of a feature extraction step of the method of FIG. 2 for the first frame of the image data according to some implementations of the present disclosure;

FIG. 6 is an illustration of a MSTMap generation step of the method of FIG. 2 for the first frame of the image data according to some implementations of the present disclosure;

FIG. 7 is a flow chart for a classical method for calculating one or more parameters for the user of FIG. 1 according to some implementations of the present disclosure; and

FIG. 8 is a flow chart for a deep learning method for calculating one or more parameters for the user of FIG. 1 according to some implementations of the present disclosure.

### DETAILED DESCRIPTION

The present disclosure provides advancements in the field of remote photoplethysmography (rPPG) as compared to prior systems by applying, for example, advanced techniques for signal extraction, peak detection and rejection, cardiovascular metrics computation, video data's inherent color channel information, along with other methods and/or steps to manage challenges posed by diverse skin tones, varying lighting conditions, motion artifacts, or any combination thereof.

According to some implementations of the present disclosure, the system includes a Plane Orthogonal to Skin (POS) Method that outperforms traditional RGB color channels in estimating heart rate accuracy. It effectively addresses issues related to skin-tone variance and luminance changes—common challenges in rPPG analysis. For example, the POS method of the present disclosure more accurately isolates photoplethysmographic signals from video data.

According to some implementations of the present disclosure, the system includes a peak detection module that is configured to determine the heart rate of a subject under different cardiac rhythms. The peak detection module can distinguish between normal sinus rhythm, premature atrial contraction, premature ventricle contraction, and atrial fibrillation. This ability significantly reduces errors in heart rate estimation and minimizes false negative peaks.

According to some implementations of the present disclosure, the system includes a proprietarily modified toolkit or a toolkit that is incorporated into the system to boost the accuracy and reliability of heart rate calculations and other vital sign measures or time series-based metrics. The toolkit accommodates amplitude variation and morphology changes of the PPG complexes. The toolkit uses a moving average and adaptive peak detection threshold to identify heartbeats. Incorrectly detected peaks are identified and rejected based on a threshold value for the RR-intervals. The toolkit also provides an optional ‘high precision mode’ that upsamples the signal for more accurate peak position estimation.

According to some implementations of the present disclosure, the system includes a comprehensive spatial and/or temporal blood metric calculation(s) for calculating a variety of time-series and frequency domain measures. These include beats per minute (BPM), interbeat interval (IBI), standard deviation of RR intervals (SDRR), standard deviation of successive differences (SDSD), root mean square of successive differences (RMSSD), proportion of successive differences above 20 ms (pNN20), proportion of successive differences above 50 ms (pNN50), median absolute deviation of RR intervals (MAD), low-frequency spectrum (LF), high-frequency spectrum (HF), and the ratio of high frequency to low frequency (HF/LF). Such comprehensive metrics provide detailed insights into an individual's health.

According to some implementations of the present disclosure, the system executes and/or includes one or more processors that are configured to execute one or more steps, such as, for example, a six-step remote vital sign measurement from one or more videos of at least a portion of a face and/or head of a user and/or subject. In some implementations of the present disclosure, the videos described herein are RGB videos. In some other implementations of the present disclosure, the videos can be and/or include IR videos or any other types of videos, or any combination thereof. In some implementations, the video or video data can also be referred to as optical or optical data, where the optical data is reproducible as images that are readable by a human (e.g., in the visible spectrum) or outside the visible spectrum. The disclosed system introduces an innovative, contactless method to measure vital signs using video data and specialized modules.

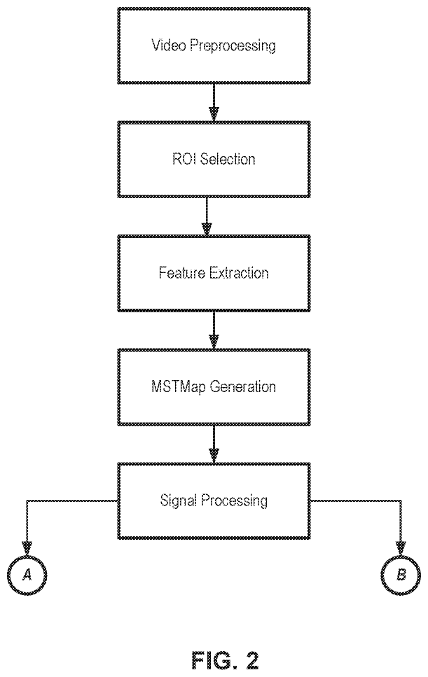

In some implementations, as shown in FIG. 2, the disclosed method is segmented into five steps: video preprocessing, region of interest (ROI) selection, feature extraction, MSTMap generation, and signal processing.

In some implementations, the disclosed method is segmented into six steps: video preprocessing, region of interest (ROI) selection, feature extraction, color intensity computation, signal processing, and post-processing, each of which is described herein in further detail below, but in brief immediately below.

In some implementations, the system begins by breaking down the video data into individual image frames for analysis. The module performs object tracking and adjusts for luminance and motion artifacts during the post-processing stage.

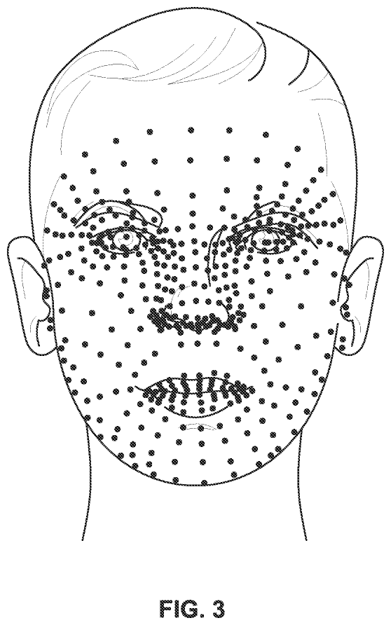

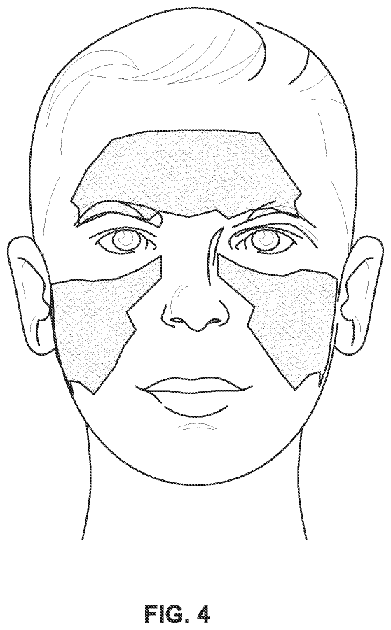

In some implementations, such as, for example, as shown in FIGS. 3 and 4, the system then identifies regions of interest within the frame, usually the subject's face. For each video frame, the system defines an ROI, taking into account the subject's movements.

In some implementations, the system then identifies face pixels within the ROI. Undesired pixels are filtered out, and the system has a built-in confidence level check for tuning parameters. The color intensity values of the extracted regions are then passed to the next step.

In some implementations, the system then determines subtle variations in the selected pixels' color intensities across the frame sequences. These color intensity changes correspond to the changes in blood volume beneath the skin, which can be correlated to heartbeats and other vital signs.

In some implementations, the system then processes the raw data to extract a clean photoplethysmography (PPG) signal. Various methods can be used individually and/or together to improve the signal-to-noise ratio and to minimize the impact of noise introduced by factors such as environmental changes or the subject's movements.

In some implementations, after obtaining the BVP signal (which can also be referred to as a clean PPG signal), the system then processes the signal further to extract physiological parameters such as heart rate, respiratory rate, heart rate variability, and oxygen saturation. This can be done, for example, by analyzing the signal's frequency components as described herein. The derived metrics can then be displayed to the user and/or transmitted to one or more third parties (e.g., a spouse of the user, a doctor of the user, etc.).

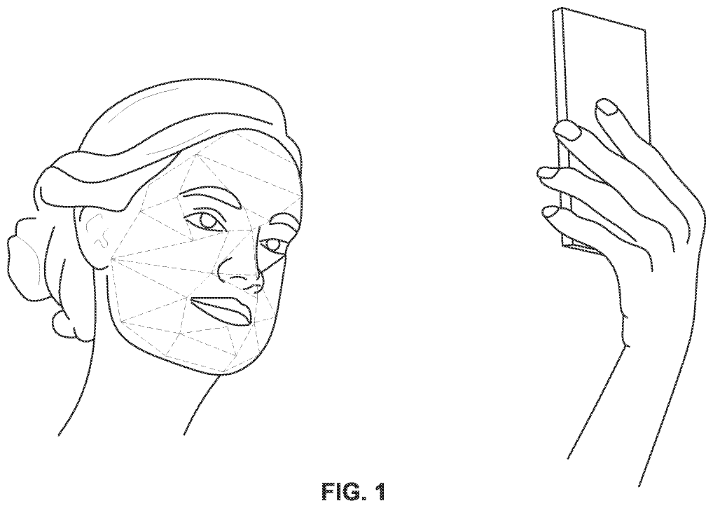

Referring to FIG. 1, a user is holding a device (e.g., a smartphone) steady and recording a video of at least a portion of a face and/or head of the user. The display of the device can include an indication of a countdown of at least five seconds on the smartphone screen to emphasize and/or aid the user in taking a video of at least 5 seconds, which will be enough content to use according to the methods disclosed herein. Any length of video can be used with the systems and methods of the present disclosure, but in some implementations, 5 seconds is preferred. In other implementations, the length of the video can be, for example, 1 second, 2 seconds, 5 seconds, 10 seconds, 30 seconds, 1 minute, etc. or any other length of time.

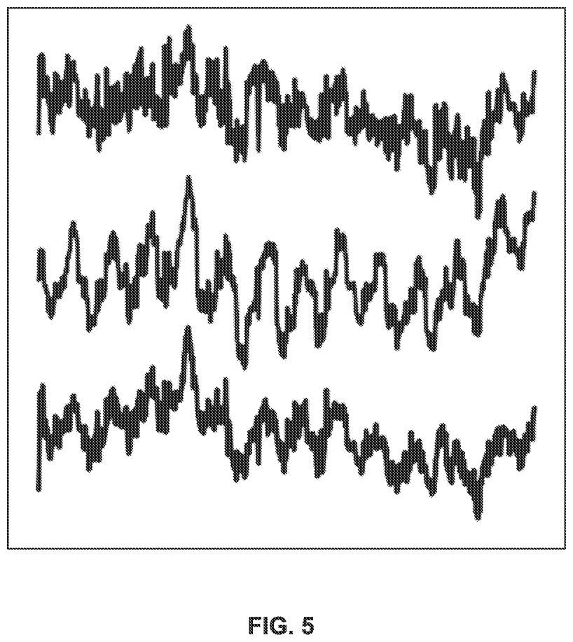

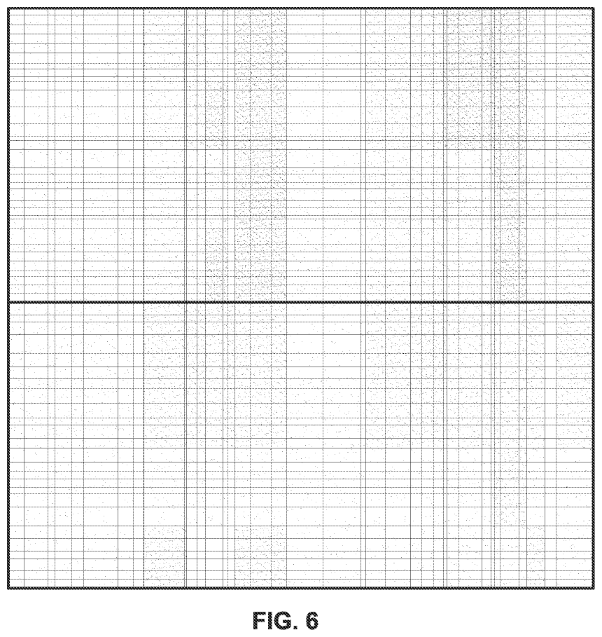

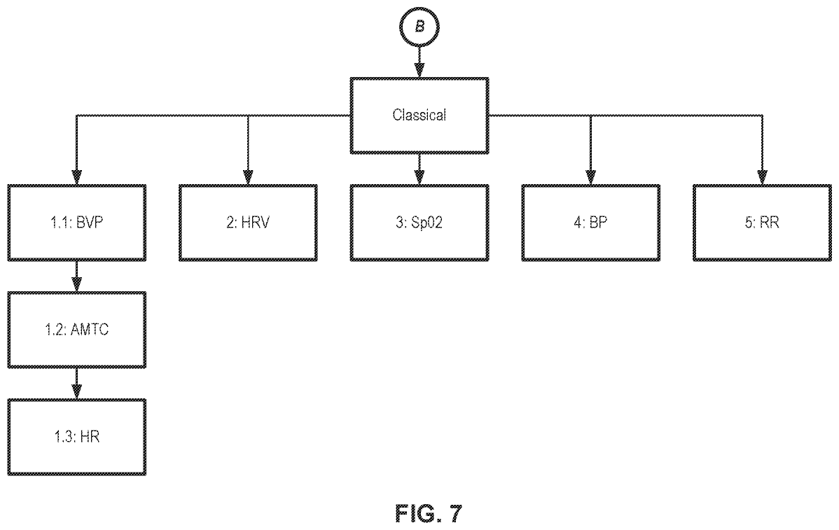

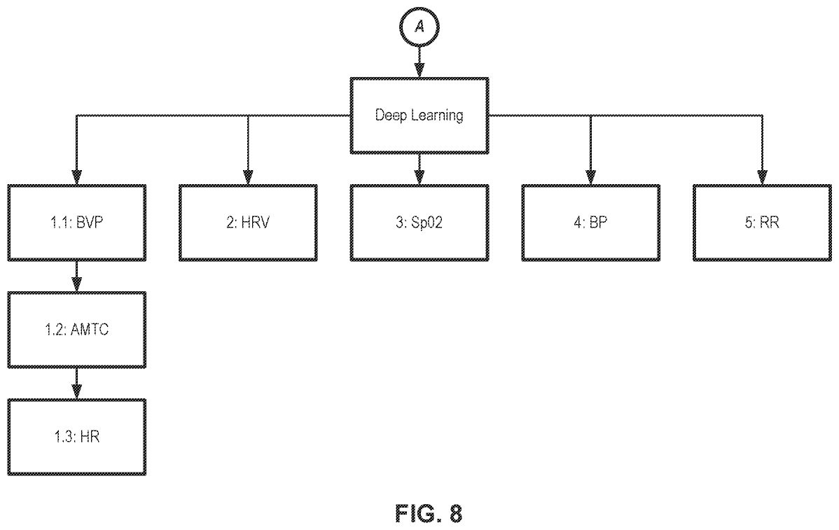

Generally referring to FIGS. 1 to 8, the present disclosure includes a video-based, contactless method of measuring vital signs using video data and specialized modules. According to some implementations of the present disclosure, the method starts with a video preprocessing step. The method/process starts with image acquisition of video content, where, for example, a high-resolution camera (e.g., at least 540p, 720p, 1080p, 4 k, 8 k, etc., or the like) and/or a smartphone camera, captures a video sample of at least a portion of the face and/or head of a user or subject or patient. In some implementations, the requirements for the camera include a spatial resolution at least 540p (resolution of 960×540), a temporal resolution of no less than 15 frames per second (fps), and a continuous video sample with a minimum duration of 5 seconds. Other implementations can use any other type of camera with advanced resolutions and/or different optical modalities. In some implementations, due to a subtle change in skin color, which correspond to changes in blood volume for the user, it is beneficial to maintain a high resolution and frame rate for the generated video content.

Once the video of the user is captured, the video is decomposed into individual frames for further processing. During this stage, a module is employed to identify a valid frames window, which can be defined as a contiguous sequence of frames with minimal interference or noise (e.g., abrupt motion and/or glaring light reflections).

Once the valid frames window is identified, the system targets a region of the image(s) that contains the face of the user and isolates some or all of skin pixels (e.g., pixels of the image that correspond to and/or represent the skin of the user's face or portion or portions of the face of the user). In some implementations, to achieve this, the system utilizes a combination of a Deep Learning (DL) network and a visual landmark tracking module. The Deep Learning network can be built upon a bilateral segmentation network (BiSeNet) that is pretrained on an internal dataset of face images of, for example, size 512×512. The system uses the DL network and the visual landmark tracking module to eliminate some or all background of the image(s) and other undesired regions of the images, such as, for example, eyes of the user, lips of the user, hair of the user, eyebrows of the user, objects in the images that might obscure the skin pixels, such as sunglasses, jewelry, and clothes, or any combination thereof.

To maintain consistency in terms of dimensions (as the DL model in some implementations may have a requirement for 512×512 images for input), the visual landmark tracking module is employed to identify the face's center, which in some implementations is the tip of the nose of the user. From this point identified center of the user's face, a 512×512 image is extracted. If the face size exceeds these dimensions, the visual landmark tracking module can be used by the system to help find the face's border coordinates such as top-most, bottom-most, left-most, and right-most, crop along those dimensions, and resize to a 512×512 image. If the frame size is less than 512, zero padding, a computer vision processing technique to maintain the image size, is applied to maintain dimension consistency.

Further, in some implementations, the visual landmark tracking module can be used to add a secondary layer of skin mask detection by identifying, for example, multiple landmarks on the face in the input frame, aiding in differentiating various face regions. These landmarks are exploited to remove eyes and lips from the input frame, and when combined with the DL mask, provide a robust and stable extraction of skin pixels from the face.

In some implementations of the present disclosure, the preprocessing stage of the system can address potential challenges such as a subject's voluntary and involuntary movements and/or environmental variations in background or lighting conditions. For this processing step, object tracking luminance correction, and signal enhancement techniques are applied to compensate for these variations.

Object tracking techniques that are implemented are from a combination of classical computer vision techniques such as canny-edge detections, affine transformations, and good features to track modules, etc. and deep learning models such as face-center and face-border tracking.

Luminance correction techniques that are implemented are from a combination of classical computer vision techniques such as color space analysis and luminance tuning and deep learning models which are primarily used to correct vertical and horizontal color imbalances and reduce signal noises.

Signal enhancement techniques that are implemented are from a combination of classical signal processing techniques such as establishing a high-pass, low-pass, and Butterworth filters and deep learning models which are used to correct signal level noises based on empirical research and internal development testing.

After these preprocessing steps, the system moves to selection of a region of interest (ROI). In this step, the system processes the masked images output from the preprocessing step to extract, in some implementations, seven (7) distinct Regions of Interest (RoIs). The RoIs are selected based at least in part on the vascular structure of the face of the user/subject. These selected RoIs are meticulously defined based on a combination of empirical research and the inventor's proprietary testing to account for potential subject movements, ensuring consistent tracking throughout the video duration.

In some implementations of the present disclosure, the system considers and/or analyzes all potential combinations of these RoIs, which results in, for example, more than 100 unique combinations. In some implementations, this results in 127 unique combinations. The combination technique increases the probability of obtaining a significant ROI, thereby ensuring reliable data for the subsequent stages of vital signs estimation. In some alternative implementations, the system considers and/or analyzes any other number of potential combinations of the RoIs resulting in relatively less unique combinations (e.g., about 5 unique combinations, about 10 unique combinations, about 50 unique combinations, about 75 unique combinations, about 100 unique combinations, about 200 unique combinations, or any other number of unique combinations using any number of RoIs.

The present disclosure's approach to RoI selection caters to diverse facial structures in a large population of potential and/or actual users, ensuring robustness and generalizability of the method(s) of the present disclosure across a wide demographic range.

After the RoIs are selected, the system then moves to feature extraction, which includes a process where the system pinpoints any number of particular pixels (e.g., facial pixels) within the selected Region of Interest (ROI) that are likely to produce precise remote Photoplethysmography (rPPG) signals.

In some implementations, for each frame in the video, the system traverses through every possible combination of regions. For each combination, the system calculates the Red (R), Green (G), and Blue (B) color intensities, taking into account the weighted average across the three-color channels, based on the number of pixels present in each region within that specific combination.

Following the extraction of the RGB intensities, in some implementations, the system then computes the Y, U, V, Cg, and Cr color space intensities by employing the following equations (rounded to third decimals):

Y=0.299*R+0.587*G+0.114*B

U=128−0.169*R−0.331*G+0.500*B

V=128+0.500*R−0.419*G−0.081*B

Cg=128−81.085*R+112*G−30.915*B

Cr=128+112*R−93.786*B−18.214*B

It should be noted that the equations provided above are but examples, and other equations, variables, or variable weights can be used in conjunction with the systems and methods provided herein. The RGB and converted color spaces are then concatenated to form a more comprehensive color feature set for each frame. In some implementations of the present disclosure, the length of each evaluation window equates to 600, the total number of region combinations equate to 127, as the system uses 7 ROIs for the feature extraction phase, the total number of color spaces equates to 9, in short, this referenced procedure in a matrix with the shape of 600×127×9 matrix.

By representing each frame with a rich set of color intensity features from multiple ROIs, the system provides an in-depth understanding of the physiological changes in the skin over time, which is usable by the system to obtain accurate vital sign estimation. The accuracy of these derived biomarkers is gauged using the inventor's proprietary dataset and/or other precedent devices developed to measure the same biomarker, consistently demonstrating a robust level of confidence under specific testing conditions.

Once the features are extracted, the system then generates one or more multi-scale spatial-temporal maps (MSTMaps). In this step, the entire matrix obtained from the previous step is subdivided into individual MSTMaps, each corresponding to a window size of, for example, about 10 seconds (or any other suitable time period greater, or shorter, than 10 seconds), which translates to 600 frames for a video running at 60 frames per second.

In some implementations, the system generates a first MSTMap from the initial 10-second segment of the video. For every subsequent MSTMap, the window is slid forward by a step equivalent to a half second or 30 frames for a 60 frames per second (fps) video. Each of these MSTMaps then undergoes a min-max normalization process and is fed individually into a deep learning neural network training pipeline as distinct inputs.

Alongside the MSTMaps, corresponding labels that contain a time-series of Blood Volume Pulses (BVP), collected from a contact device during data collection process, are divided based on the same window size. In some implementations, a BVP is partitioned for the 10-second window. If the sampling rate of the sensor differs from the video's frame rate (in this implementation, 60 fps), the BVP is interpolated to ensure that it aligns with the MSTMap's window length.

Similarly, in some implementations, a beats per minute (BPM) is split based on the window size. For each MSTMap, an average BPM value can also be calculated, which serves as the ground truth BPM for that MSTMap.

Through this process, each MSTMap and its associated ground truth BPM are distinctly tied to a specific segment of the video, effectively allowing the training process to learn the relationship between the input features and the target vital signs on a segment-by-segment basis.

Once the MSTMap(s) are generated, the system then utilizes the preprocessed MSTMaps to estimate various vital signs, including Heart Rate (HR), Heart Rate Variability (HRV), Oxygen Saturation (SpO2), and Respiratory Rate (RR). The process consists of several sub-steps.

In some implementations, the system uses a Deep Learning Approach (DLA). In DLA, the system starts with an rPPG Estimator, which includes a deep neural network that takes raw preprocessed MSTMaps and produces predicted signals useful for the estimation of HR, HRV, SpO2, and RR. The DLA's network architecture comprises several layers of attention blocks and base blocks, followed by a sequence of upsampling blocks. After hyperparameter tuning, in some implementations, the drop-out rate is set to 0.2.

In some implementations, the system includes a loss function, a mathematical function that helps the deep learning model to obtain feedback on how good/poor the model has performed based on a pre-defined function. In some of the implementations, loss function is defined as a weighted combination of a Negative Pearson's Correlation and a Cross Signal-to-Noise Ratio (SNR). The Negative Pearson's Correlation is used to capture a linear similarity between the ground truth and a predicted signal, while the Cross SNR enhances signal robustness by reducing the impact of noise. In this loss function, a proprietary allocation of weights can be assigned to Pearson's correlation and SNR. In some implementations, the system is trained for at least about 72 hours on, for example, a Tesla T4 GPU with a starting learning rate of 0.01, using a StepLR scheduler with step size of 4 and gamma of 0.5, in batches of 8 MSTMaps. The system that offers the lowest root mean square error (RMSE) on BPM based on a predefined proprietary validation set, and every tenth epoch model, are saved. All saved models are evaluated on the validation set to identify the deployed model for future use.

In some implementations of the present disclosure, the system includes a Decomposition and Reconstruction Network (DRNet) model to determine and/or provide a Blood Volume Pulse (BVP) as an output which is used for the heart rate estimation. The BVP signals determined from consecutive MSTMaps are merged by an Adaptive Multi-Trace Carving (AMTC) Module. Such merging can be achieved using a weighted average approach, where weights depend on the prediction's position within the window and the number of other predictions containing the frame. A triangular weighted function is applied to the BVP signals, assigning maximum weight to the signal at the frame's center, with weights decreasing as the signal moves away from the center.

The Adaptive Multi-Trace Carving (AMTC) Module is assigned specific variables and functions as shown in the equations below. In some implementations, the AMTC Module includes a discrete function where “n” is defined as [0, l−1] and “l” is the length of the window. This function gives a weight to the prediction with respect to its position on the window. “S” is the step size for the moving window processing.

$$( n ) = \{ \begin{matrix}w ( n - i \cdot s ) , & if i \cdot s \leq n < i \cdot s + l \\ 0 , & otherwise\end{matrix}$$

Which is simply the w shifted in time.

$$( n ) = \{ \begin{matrix}1 , & if n = 0 or n = t f - 1 \\ {\sum }_{r = 0}^{N - 1} , & otherwise\end{matrix}$$

Where tf=total number of frames
Then the weight for each prediction is

$${w}_{i} ( n ) = \{ \begin{matrix}w ( n - i \cdot s ) / W ( n ) , & if i \cdot s \leq n < i \cdot s + l \\ 0 , & otherwise\end{matrix}$$

Let pᵢ be the rPPG predicted form window i then:

$${P}_{i} ( n ) = \{ \begin{matrix}{P}_{i} ( n - i \cdot s ) , & if i \cdot s \leq n < i \cdot s + l \\ 0 , & otherwise\end{matrix}$$

These functions are for any integer n that represents the index of a frame of the video. So, n is less than or equal to the total number of frames. The average rPPG signal is given by:

$$P ( n ) = {\sum }_{r = 0}^{N - 1} {ω}_{r} ( n ) {P}_{r} ( n )$$

The resulting averaged rPPG signal (P(n)) is then processed by the AMTC Module, which tracks frequency traces. The system outputs information and/or data designed to handle signal noise and assumes the changing frequency of the heart rate to be continuous. In some implementations, the system focuses on tracking a single frequency trace. The loss function combines a component favoring the highest energy frequency and a component based on the Transition Probability Matrix. This matrix represents the probability of the next predicted BPM (y-axis) given the previous BPM (x-axis), based on a normal distribution with a mean (μ) of the previously detected frequency and a standard deviation (σ) of 0.3, as determined by experimental findings. In some implementations, the output of the AMTC Module provides BPM values for the video, representing the heart rate of the user/subject.

In some implementations, the BVP signal generated by the DRNet model is input into an HRV processing module. The HRV processing module is configured to yield a raw time series that includes both accepted and rejected peaks. Through a series of signal processing, purifying steps, and thresholding, a cleaner signal time-series is obtained. The occurrence times of heartbeats are identified, and the inter-beat interval (IBI) is computed. Further calculations include time-series measurements such as beats per minute (BPM), inter-beat interval (IBI), standard deviation of NN intervals (SDNN), standard deviation of RR intervals (SDRR), root mean square of successive RR interval differences (RMSSD) and more. Frequency domain measures can also be determined by the system. The raw HRV value is calculated over a 30-second window with a step size of 1 second. According to some implementations, the system can provide and/or output a mean absolute percentage error (MAPE)-based accuracy of 78% when using a signal confidence threshold of 90%.

In some implementations, the following HRV metrics are considered in the post-processing evaluation by the system:
SDNN (ms): Standard deviation of NN intervals
SDRR (ms): Standard deviation of RR intervals
RMSSD (ms): Root mean square of successive RR interval differences

In some implementations, the MSTMaps can be used by the system to measure blood oxygen levels in terms of SpO2. Similar to the HR estimation, the MSTMaps are input into the DRNet model, but in this case, SpO2 values are used as labels. This allows the model to learn the underlying principles of absorption of different wavelengths by deoxygenated and oxygenated blood, aiding in SpO2 estimation.

In some implementations, blood pressure can be estimated using transdermal optical imaging (TOI), which is a variant of rPPG. This technique detects blood flow pulses by comparing the re-emitted light during systolic and diastolic cycles. The variation in light intensity due to changes in blood volume is used to estimate Pulse Transit Time (PTT), a measure commonly used as an indicator for blood pressure.

In some implementations, a BVP signal generated by the DRNet model is used to estimate the Respiratory Rate (RR) using a similar process to that used for HRV estimation. The RR processing module produces a raw time series containing both accepted and rejected peaks. Following several signal processing, purifying steps, and thresholding, a clean signal time-series is obtained and processed. The output of the RR processing module can provide a rough estimation of the respiratory rate. The raw RR value is then calculated over a 30-second window with a step size of 1 second.

In some implementations of the present disclosure, the system uses a classical approach that employs the MSTMaps as described herein to extract the R, G, B signal from these MSTMaps. Instead of the YUV color space, this classical approach uses the YCgCr color space. In some such implementations, the green channel in rPPG is utilized for heart rate extraction, but luminance changes can interfere with this extraction. The YCgCr color space can be used to mitigate this interference by separating luminance from color channels. The Cg and Cr signals undergo additional preprocessing to reduce noise and other artifacts.

From the YCgCr signal, the module stored Y component for post-processing in step 4 and the components of interest in this step are the Cg and Cr. Once the Cg and Cr signals are obtained, there often remains noise due to various artifacts. These artifacts can be corrected using signal preprocessing such as some type of bandpass filter (e.g., Bessel or Butterworth).

Since both the green channel and the Cg channel values from the camera are depending on the skin tone of the subject, these channels could be heavily correlated with the noise from changes on the light conditions due to movement of the subject or changes from the light source. The Plane Orthogonal to Skin (POS) proposed tries to find a projection of the RGB colors in the direction of the heart rate signal. This is achieved by the following steps:
1. Calculate R_{N}, G_{N}, B_{N} as the zero-mean-scaled and filtered time series of the color channels RGB.
2. Compute and obtain: X_{S}=G_{N}−B_{N}, Y_{S}=−2R_{N}+G_{N} B_{N}
3. Estimate σ=σL(X_{S})/σL(Y_{S}) as the rolling standard deviation of X_{S} over the rolling standard deviation of Y_{S}. Both rolling standard deviations use L points of the time series to be calculated. L is the number of samples necessary to cover at least one heartbeat.
4. Finally, compute S_{POS}=X_{S}+σY_{S} as the channel that will be used to estimate the heart rate in a next step.

S_{POS} signal is used to estimate the heart rate. In some implementations, the system uses a peak finding method in the time domain. The heart rate is calculated by finding the difference in time in seconds between each peak. The peaks of the signal are found at time t₀ and t₁. Then, the heart rate is equal to 60/(t₁−t₀). The estimation of the heart rate will be the same for any value between t₀ and t₁.

In some implementations, the system uses a peak finding method in the frequency domain. Heart rate is estimated by using the Fourier transform in sections of the signal.

In some implementations, a sliding window approach is used where a portion of the signal is processed. The Fourier transform is applied on the portion of the signal and the magnitude of the signal in the frequency domain is calculated. The frequency that corresponds to the highest magnitude is selected as the estimation of the heart rate of the central time of the window. As an example, for the first window from the image, the central time of the window is t₀ and the selected frequency is f₀.

In some implementations, the system uses a signal confidence evaluating technique. One common challenge when applying Fourier transform to obtain peak signal for the heart rate is that the correct signal could be hidden from the noise signals. In some but not rare instances, the noise signals are overwriting the correct signals and resulting jump behaviors in the raw calculated results. To address this issue, the module takes advantage of the fact that the analyzed video is a continuous series of frames and is able to compare, monitor and evaluate the migration of signal strengths to filter out outlier signal frames. For example, one of the evaluation processes is to compute the ratio between two adjacent signal peaks and evaluate the likelihood of migration. The module applies a series of signal processes to confirm if the migration is attributed from actual heart rate change or false readings from the noise signals.

In some implementations of the present disclosure, a POS module determines and outputs a BVP which can be fed into the AMTC module described above to get the BPM.

In some implementations of the present disclosure, the system implements time series measurements, such as heart rate variability (HRV). The BVP signal generated by the POS model is input into the HRV processing module. The module yields a raw time series that includes both accepted and rejected peaks. Through a series of signal processing, purifying steps, and thresholding, a cleaner signal time-series is obtained. The occurrence times of heartbeats are identified, and the inter-beat interval (IBI) is computed. Further calculations include time-series measurements such as beats per minute (BPM), inter-beat interval (IBI), standard deviation of NN intervals (SDNN), standard deviation of RR intervals (SDRR), root mean square of successive RR interval differences (RMSSD) and more.

In some implementations of the present disclosure, the system implements frequency domain measurements. Such frequency domain measurements can be used to examine a power distribution of the IBI series as a function of frequency. This distribution can be divided into different bands, such as low-frequency (LF) and high-frequency (HF) bands. The LF band provides information about both sympathetic and parasympathetic activity, whereas the HF band is primarily indicative of parasympathetic activity.

In some implementations of the present disclosure, the system implements an oxygen saturation measurement. In such oxygen saturation measurements, the oxygen saturation level is estimated using a ratio-of-ratios method, which employs the Beer Lambert law and compares the absorption of different wavelengths by deoxygenated and oxygenated blood. The system evaluates the relative absorption characteristics of two light wavelengths, one absorbed more by deoxygenated hemoglobin (isosbestic wavelength) and the other absorbed more by oxygenated hemoglobin. The ratio of these two measures provides the SpO2 estimation.

In some implementations of the present disclosure, blood pressure calculation and processing is done using transdermal optical imaging (TOI) technique which is a variant of remote photoplethysmography (rPPG). This technique capitalizes on subtle changes in skin color from the difference in the re-emitted light between hemoglobin and melanin chromophores to detect blood flow pulsation. The process of blood pressure estimation can be broken down to the following steps: (1) video recording of the face is used to capture the light re-emitted by blood hemoglobin; (2) this re-emitted light is inversely proportional to hemoglobin concentration near the skin surface which requires signal processing to obtain BP signals; and (3) post the signal processing, the module extracts many features including pulse amplitude, heart rate band pulse amplitude, pulse rate, pulse rate variability, pulse transit time, pulse shape, and pulse energy among others.

Uses signal processing to extract hemoglobin rich signal and discard melanin-rich signal from each frame of the video. These hemoglobin signals are combined to produce an image representing the concentration of hemoglobin across the face. The changing hemoglobin concentration across frames represents blood flow oscillations. Upon the signal processing and feature extraction, as mentioned above, the module performs principal component analysis (PCA) to reduce the feature dimension spaces. The results from this step are passed into BioEngine4D's machine learning model to obtain the following vital health statistics: (1) systolic blood pressure; (2) diastolic blood pressure; and (3) pulse pressure.

The BVP signal generated by the POS model is used to estimate the Respiratory Rate (RR) using a similar process to that used for HRV estimation. The RR processing module produces a raw time series containing both accepted and rejected peaks. Following several signal processing, purifying steps, and thresholding, a clean signal time-series is obtained and processed. The output of the model provides a rough estimation of the respiratory rate. The raw RR value is then calculated over a 30-second window with a step size of 1 second.

In some implementations of the present disclosure, the system implements one or more post-processing steps. For example, the system can include post-processing steps such as an outlier removal based on human physiological constraints, smoothing changes in heart rate, oxygen saturation, respiration rate, and/or blood pressure, interpolating values that were removed in the previous steps, and any combination thereof. The effectiveness of these post-processing steps can be assessed by comparing the root mean squared error (RMSE) of various heart rate data: heart rate, motion-adjusted heart rate, luminance-adjusted heart rate, motion and luminance-adjusted heart rate, and post-processing heart rate, or any combination thereof.

Any combination of one or more of the steps disclosed herein can be used to enhance the accuracy and precision of measured vital signs from image and/or video data, particularly in non-controlled videos with artifacts introduced by subject motion, environmental lighting, and other dynamic factors.

According to some implementations of the present disclosure, the Deep Learning Approach (DLA) can be trained on a dataset of approximately two hundred 60 fps videos, with 80 videos used for training and 20 used for intra-dataset evaluation, in a controlled environment with minimal movement. This setup can allow the system to extract an rPPG signal from the input videos effectively and provide a reliable estimate of BVP and, consequently, the Heart Rate. The DLA of the present disclosure can also be evaluated using another dataset under various lighting conditions both before and after physical activity. For the intra-dataset evaluation, in some implementations of the present disclosure, the system achieved a mean absolute error (MAE) of 0.51 BPM and a root mean square error (RMSE) of 1.23 BPM. For the cross-dataset evaluation, the system achieved an MAE of 5.38 BPM and RMSE of 7.20 BPM on the test set.

According to some other implementations of the present disclosure, the system achieves an MAE between about 0.01 BPM and about 8 BPM and a RMSE between about 0.1 BPM and about 15 BPM. According to some other implementations of the present disclosure, the system achieves an MAE between about 0.1 BPM and about 4 BPM and a RMSE between about 0.5 BPM and about 5 BPM.

In some implementations of the present disclosure, the system employs a confidence weighting strategy to enhance the accuracy and/or precision of vital signs estimation on a frame-by-frame basis. Within this framework, the system may incorporate the “Motion Artifact Confidence Weight” (MACW) approach, which addresses noise introduced by the subject's movements; the “Luminance Artifact Confidence Weight” (LACW) approach, which mitigates noise resulting from variations in background luminance; the “Data Fusion” (DF) approach, which amalgamates inferences from both deep learning and classical methodologies to bolster estimation robustness; or a synergistic combination of these methodologies.

In some implementations, the system adopts a frame-by-frame inference confidence approach, specifically weighted against motion artifacts, termed “Motion Artifact Confidence Weight” (MACW). This strategy is designed to address and neutralize the noise introduced by the subject's movements during the video capture process. Such movements, whether subtle or pronounced, can introduce discrepancies in the data, potentially affecting vital sign estimations. To address this challenge, the system incorporates a dedicated module, fine-tuned to detect and evaluate motion artifacts. This system employs advanced modules and heuristics to discern motion-induced disturbances within the video data. By analyzing the temporal and spatial characteristics of the video frames, it can pinpoint areas where motion artifacts are prevalent. Upon the identification of these artifacts, the system proceeds to assign confidence weights to each data point within the estimated vital signs time series. These weights serve as indicators of the data point's accuracy in relation to motion artifacts. The process of weight determination is based on a proprietary formulation, meticulously crafted to consider various factors. This formulation takes into account both the nature of the identified motion artifacts (e.g., abrupt vs. gradual movements) and their amplitude (e.g., magnitude of motion).

In some implementations, the system employs a frame-by-frame inference confidence approach tailored against luminance artifacts, termed “Luminance Artifact Confidence Weight” (LACW). This approach is fundamentally designed to mitigate the potential distortions arising from fluctuations in the background luminance during video capture. Such fluctuations can be attributed to a myriad of factors, including changes in ambient lighting, reflections, or even the subject's proximity to light sources. The system is equipped with a specialized module, adept at detecting and quantifying these luminance discrepancies within the video data. By leveraging advanced modules and computational techniques, this module can discern variations in luminance that might otherwise go unnoticed yet have a profound impact on the accuracy of vital sign estimations. Once these luminance artifacts are identified, the system introduces a rectification factor to the inference. This factor serves to adjust and correct the signal, ensuring that the derived vital signs are not skewed by the identified luminance variations. The formulation of the rectification factor is based on a proprietary module, which has undergone rigorous optimization processes. This ensures that the LACW approach is adept at handling a wide spectrum of luminance artifacts, be they subtle shifts in ambient lighting or pronounced changes due to direct light exposure.

In some implementations, for the Data Fusion approach, the system synergistically integrates the insights derived from both the Deep Learning Approach (DLA) and the Classical Approach to independently estimate the vital signs. This amalgamation is designed to harness the strengths of both methodologies, thereby aiming to bolster the precision and reliability of the vital sign estimations. Specifically, the Deep Learning Approach (DLA) leverages intricate neural network architectures to discern patterns and relationships within the data that might be imperceptible or less evident to classical modules. The DLA is trained on vast datasets, enabling it to generalize and predict vital signs even under varying conditions.

V_DLA=f_DL(D), Where:
V_DLA is the vital sign estimation using the Deep Learning Approach.
f_DL represents the deep learning function.
D is the input data.

Conversely, the Classical Approach is rooted in established physiological and mathematical models, offering a deterministic perspective to vital sign estimation. It employs signal processing techniques and predefined modules to extract and interpret the relevant physiological signals from the data.

V_CA=f_C(D) Where:
V_CA is the vital sign estimation using the Classical Approach.
f_C represents the classical function.
D is the input data.

In the Data Fusion approach, the system is configured to compute a weighted average of the estimations from both DLA and the Classical Approach, optimizing the final output. The weights can be dynamically adjusted based on the confidence levels or the environmental conditions to ensure optimal performance.

V_DF=α*V_DLA+(1−α)*V_CA Where:
V_DF is the final vital sign estimation using the data fusion approach.
α is a weighting factor between 0 and 1.

This fusion not only capitalizes on the predictive prowess of deep learning but also grounds the estimations in the reliability and interpretability of classical modules. The combined approach aims to deliver enhanced robustness, especially in scenarios where one method might falter, ensuring consistent and accurate vital sign estimations across a myriad of conditions.

In some implementations of the present disclosure, the system undertakes additional post-processing measures. These entail the excision of outliers rooted in human physiological bounds, the attenuation of aberrations in heart rate, oxygen saturation, respiration rate, and/or blood pressure, and interpolation of values discarded during prior stages. The efficacy of such post-processing actions is evaluated by contrasting the root mean squared error (RMSE) across various heart rate datasets: pre-processed heart rate, motion-compensated heart rate, luminance-corrected heart rate, heart rate adjusted for both motion and luminance, and the post-processed vital sign in question.

Building on the foundations established by the methodologies described above, some future implementations of the system of the present disclosure include an end-to-end neural network system. Such implementations transition from the segmented process of data pre-processing feeding into an AI or neural net, followed by post-processing to infer vital sign values. This approach aims to directly convert raw video data, which may include face or other physiological indicators, into vital sign values. A distinctive innovation in this approach lies in the generation of a training dataset using our existing classical-neural network-classical (CNC) method. By doing so, the end-to-end neural network can learn the entire process, potentially streamlining and enhancing the efficiency of vital sign estimation. The method of generating the training data for this end-to-end neural network forms a pivotal component of this advancement, especially given the challenges in creating accurate labels, such as vital signs, at scale—a task that's more complex than just generating face video data.

According to some implementations of the present disclosure, one or more of the steps disclosed herein can be used by the system to improve and/or enhance the accuracy and precision of measured vital signs form images and/or video data, even under the challenges posed by non-controlled environments and dynamic contexts. While some challenges may arise in uncontrolled environments, the system is resilient and has improved robustness compared to prior systems when evaluating images and video. In particular, the systems of the present disclosure analyze pre-recorded and/or retrospectively captured videos effectively, allowing the system to extract biomarkers from them, including biomarkers from older videos (e.g., videos captured during or after the year 1990, during or after the year 2000, during or after the year 2010, during or after the year 2020, etc. or any other prior year of capture). The ability of the system of the present disclosure is an improvement in technology as compared to prior systems as the systems of the present disclosure unlock the potential to derive valuable longitudinal health and/or instant behavioral-modifying vital signs information from a broad range of video sources, prospectively and retrospectively, making the system highly versatile and impactful in the fields of healthcare and beyond.

### Alternative Implementations

Alternative Implementation 1. A remote photoplethysmography (rPPG) system that captures RGB data from video frames for vital sign extraction, the video frames are derived from a user-recorded video selfie utilizing a mobile device's camera, thus providing an innovative method of health measurements.

Alternative Implementation 2. The system of Alternative Implementation 1, which utilizes RGB data to generate a photoplethysmograph (PPG)-like signal using one or more of the present disclosure's proprietary modules, POS, using a projection plane orthogonal to the skin for pulse extraction, distinguishing the technique from traditional methods.

Alternative Implementation 3. The system of Alternative Implementation 1 or 2, which applies normalization, filtering, and signal quality assessment to the PPG-like signal, reducing noise and artifacts, thus enhancing the precision of vital sign extraction.

Alternative Implementation 4. The system of any preceding Alternative Implementation, wherein the processed PPG-like signal is analyzed to identify individual heartbeats, using adaptive thresholding, a moving average, and outlier detection and rejection, providing more nuanced heart rate measurements beyond standard hemoglobin concentration changes.

Alternative Implementation 5. The system of any preceding Alternative Implementation, capable of extracting heart rate, heart rate variability, oxygen saturation, respiration rate, and blood pressure from the PPG-like signal, offering comprehensive health insights not typically provided by standard video-based health tracking systems.

Alternative Implementation 6. The system of any preceding Alternative Implementation, which applies a sequential series of modules to discriminate various arrhythmias based on the extracted health features, going beyond standard vital sign measurements to acute health monitoring.

Alternative Implementation 7. The system of any preceding Alternative Implementation, which employs a PPG peak detection method that reduces heart rate estimation errors by minimizing false negative peaks, improving the accuracy of heart rate tracking beyond standard methods.

Alternative Implementation 8. The system of any preceding Alternative Implementation, which integrates a high precision mode into the module that upsamples the signal for more accurate peak position estimation, ensuring precise vital sign readings.

Alternative Implementation 9. The system of any preceding Alternative Implementation, which computes both time-series and frequency domain measures from the detected peaks, offering a robust analysis of the user's health data.

Alternative Implementation 10. The system of any preceding Alternative Implementation, where the module's mean absolute percentage error (MAPE) outperforms other color channels in controlled environments, showing the system's superior performance.

Alternative Implementation 11. The system of any preceding Alternative Implementation, which expands the system's functionality by estimating the heart rate using a peak finding method in the frequency domain for all methods, except when using the POS AMTC approach, which applies the AMTC method on the POS signal.

Alternative Implementation 12. The system of any preceding Alternative Implementation, which allows clinicians to leverage the extracted PPG data for more accurate heart rate control recommendations, going beyond mere heart rate tracking.

Alternative Implementation 13. The system of any preceding Alternative Implementation, where the output is used for wellness and health trend monitoring, supplementing professional medical advice, and not intended as a replacement.

Alternative Implementation 14. The system of any preceding Alternative Implementation, where a user records a video selfie, as motionless as possible, for a short duration and repeats this process regularly to monitor changes in their vital signs over time, to aid the user in a continuous health monitoring.

Alternative Implementation 15. The system of any preceding Alternative Implementation, which adjusts for variations in illumination during the video selfie recording under specified illumination conditions, addressing potential challenges in different lighting conditions.

Alternative Implementation 16. The system of any preceding Alternative Implementation, which adjusts for variations in the quality and specifications of different front-facing cameras used for the video selfie, ensuring compatibility across a range of devices.

Alternative Implementation 17. The system of any preceding Alternative Implementation, which excludes potentially signal-disruptive facial features such as eyes, lips, and facial hair from the analysis, thereby improving the accuracy of the rPPG signal, the BVP signal (e.g., a clean version of the rPPG signal), or both.

Alternative Implementation 18. The system of any preceding Alternative Implementation, which incorporates machine learning models to adjust for potential skin tone variations across different users, ensuring robust.

Alternative Implementation 19. The system of any preceding Alternative Implementation, wherein the system includes an onboard user interface on the user's mobile device for real-time visual display of the extracted vital signs and historical health trends, to aid in fostering awareness and management of the user's health.

Alternative Implementation 20. The system of any preceding Alternative Implementation, wherein the system integrates with third-party health and wellness applications, thereby expanding the scope of digital health management by sharing and synchronizing health data.

Alternative Implementation 21. The system of any preceding Alternative Implementation, which uses secure data storage and transmission protocols, ensuring the privacy and safety of the user's health data.

Alternative Implementation 22. The system of any preceding Alternative Implementation, wherein the user's data is anonymized and pooled with other user data for large-scale health trend analysis, contributing to broader health research while maintaining individual privacy.

Alternative Implementation 23. The system of any preceding Alternative Implementation, wherein the system sends notifications and reminders to the user to record video selfies, ensuring regular health measurements.

Alternative Implementation 24. The system of any preceding Alternative Implementation, wherein the system generates user-specific recommendations based on the analysis of vital signs and health trends, thereby aiding in promoting personalized healthcare.

Alternative Implementation 25. The system of any preceding Alternative Implementation, which has an option to notify healthcare professionals or caregivers in case of significant changes in the user's health, enabling timely medical intervention.

Alternative Implementation 26. The system of any preceding Alternative Implementation, which can adapt its module to cater to different demographics or environmental conditions, including age, sex, skin tone, health conditions, lighting and/or motion, ensuring more customized and accurate measurements.

Alternative Implementation 27. The system of any preceding Alternative Implementation, which includes a feedback loop from users and healthcare professionals for continuous system improvement, fostering an evolving health monitoring solution.

Alternative Implementation 28. The system of any preceding Alternative Implementation, where the present invention can partner with healthcare providers and insurers to offer this technology as part of a broader healthcare package, broadening its reach and impact.

Alternative Implementation 29. The system of any preceding Alternative Implementation, which also includes the capability to detect subtle changes in user's physiological and emotional states by analyzing skin and other facial features, enhancing its functionality.

Alternative Implementation 30. The system of any preceding Alternative Implementation, wherein the rPPG technology of the system, along with one or more of the other system features, is contained within an application software for mobile devices.

1. A method for determining one or more physiological parameters of a subject, the method comprising:
generating video data, the video data being reproducible as a plurality of frames, each of the plurality of frames depicting at least a portion of the subject;
for each of the plurality of the frames, identifying a region of interest;
for each region of interest in each of the plurality of frames, extracting pixels;
determining color intensity variations for at least a portion of the pixels across at least a portion of the plurality of frames; and
using the determined color intensity variations, determining the one or more physiological parameters for the subject.

2. The method of claim 1, wherein the method is performed using a remote photoplethysmography (rPPG) system, and wherein the video data is generated using one or more cameras of a mobile device associated with the subject.

3-4. (canceled)

5. The method of claim 1, to wherein the identifying the region of interest includes accounting for a subject's movements and excluding potentially signal-disruptive facial features, wherein the potentially signal-disruptive facial features include eyes of the subject, lips of the subject, facial hair of the subject, or any combination thereof.

6. (canceled)

7. The method of claim 1, further comprising filtering, from the pixels, undesired pixels based, at least in part, on a confidence level check.

8. The method of claim 1, wherein the generated video data has a resolution about 540p and the video data was generated at least about 1 year prior to the determining the one or more physiological parameters for the subject.

9. The method of claim 1, wherein the color intensity variations of the pixels are determined via a plane orthogonal to skin (POS) method, and wherein the POS method compensates for subject motion, skin variance, ambient light changes, or any combination thereof.

10. (canceled)

11. The method of claim 1, wherein the one or more physiological parameters include blood volume pulse (BVP), heart rate (HR), heart rate variability (HRV), oxygen saturation (SpO2), respiration rate (RR), and blood pressure (BP), interbeat interval (IBI), standard deviation of RR intervals (SDRR), standard deviation of successive differences (SDSD), root mean square of successive differences (RMSSD), proportion of successive differences above 20 ms (pNN20), proportion of successive differences above 50 ms (pNN50), median absolute deviation of RR intervals (MAD), low-frequency spectrum (LF), high-frequency spectrum (HF), a ratio of high frequency to low frequency (HF/LF), or any combination thereof.

12. The method of claim 1, wherein the generated video data was generated at least about 1 day prior to the determining the one or more physiological parameters for the subject.

13-14. (canceled)

15. The method of claim 1, further comprising causing presentation, via a mobile device, one or more vital signs of the subject, contextual information associated with the one or more vital signs, historical health trends of the subject, comparisons of the health trends of the subject with others, or any combination thereof.

16-17. (canceled)

18. The method of claim 1, further comprising:
causing a reminder notification to be presented to the subject to remind the subject to record a video;
generating, based at least in part on the one or more physiological parameters, recommendations for the subject;
determining that at least one of the one or more physiological parameters has changed an amount above a predetermined threshold for the subject;
generating, based on the determined change, a notification; and
automatically transmitting the notification to a computer system associated with a healthcare provider.

19-20. (canceled)

21. The method of claim 1, wherein the determining the one or more physiological parameters includes accounting for an age of the subject, a sex of the subject, a skin tone of the subject, health conditions of the subject, lighting in the video data, motion in the video data, or any combination thereof.

22. The method of claim 1, further comprising:
analyzing the skin of the subject, the facial features of the subject, or both; and
detecting, based at least in part on the analyzing, changes in a physiological state of the subject, an emotional state of the subject, or both.

23-24. (canceled)

25. The method of claim 1, wherein the region of interest includes at least two regions of interest, and wherein the at least two regions of interest are noncontiguous.

26. (canceled)

27. The method of claim 1, further comprising:
performing signal processing on the color intensity variations to extract blood volume pulses (BVP), wherein the BVP is extracted via (i) a signal extraction and peak detection module (ii) a rejection module to minimize the impact of one or both of noise and motion artifacts, or (iii) both (i) and (ii), wherein the signal extraction and peak detection module compensates for amplitude variation and morphology changes of photoplethysmographic complexes.

28-29. (canceled)

30. The method of claim 1, wherein the video data includes RGB data and the method further comprises:
generating, based on the RGB data, a photoplethysmograph-like (PPG-like) signal; and
determining pulse extraction via a projection plane orthogonal to skin of the subject.

31-49. (canceled)

50. A method for determining one or more physiological parameters of a subject, the method comprising:
generating video data, the video data being reproducible as a plurality of frames, each of the plurality of frames depicting at a portion of the subject;
stabilizing, via a motion artifact confidence weight (MACW) module, each of the plurality of frames;
processing, via a luminance artifact confidence weight (LACW) module, each of the plurality of frames; and
estimating, via one or both of a classical approach module and a deep learning network module, the one or more physiological parameters of the subject.

51. The method of claim 50, wherein the estimating the one or more physiological parameters of the subject includes both the classical approach module and the deep learning network module, and wherein the method further comprises integrating data derived from the classical approach module and data derived from the deep learning network module.

52. The method of claim 50, wherein the MACW module accommodates for movement of the subject during a capture of the generated video data, wherein the processing each of the plurality of frames includes quantifying luminance discrepancies within the video data, wherein the processing the plurality of frames includes introducing a rectification factor associated with the luminance discrepancies.

53-54. (canceled)

55. A method for determining one or more physiological parameters of a subject, the method comprising:
generating video data, the video data being reproducible as a plurality of frames, each of the plurality of frames depicting at least a portion of the subject;
stabilizing, via a motion artifact confidence weight (MACW) module, each of the plurality of frames;
processing, via a luminance artifact confidence weight (LACW) module, each of the plurality of frames;
for each of the plurality of the frames, identifying a region of interest;
for each region of interest in each of the plurality of frames, extracting pixels;
determining color intensity variations for at least a portion of the pixels across at least a portion of the plurality of frames; and
using the determined color intensity variations, determining the one or more physiological parameters for the subject.

56. The method of claim 55, wherein the determining the one or more physiological parameters includes estimating, via one or both of a classical approach module and a deep learning network module, the one or more physiological parameters of the subject.

57. (canceled)
